(19) United States
(12) Patent Application Publication
(10) Pub. No.: US 2025/0241779 A1
(43) Pub. Date: Jul. 31, 2025

(54) BRACE DEVICE

(71) Applicant: Jieqiong ZHU, Beijing (CN)

(72) Inventor: Jieqiong ZHU, Beijing (CN)

(21) Appl. No.: 19/080,382

(22) Filed: Mar. 14, 2025

Related U.S. Application Data
(63) Continuation of application No. PCT/CN2023/124226, filed on Oct. 12, 2023.

(30) Foreign Application Priority Data
Oct. 13, 2022 (CN) 202222696141.4

(51) Int. Cl. A61F 5/01

(52) U.S. Cl. CPC A61F 5/0193

(57) ABSTRACT

A brace device includes: a first brace for fixing a calf and a foot in the circumferential direction of the calf; a second brace for receiving a thigh, and generally kept fixed relative to a body; and a pivotable connection assembly having one end fixedly connected to the first brace and the other end fixedly connected to the second brace. Taking the position of the pivot axis of the connection assembly when the tiptoe points straight ahead as a reference, the mounting position of the connection assembly on the second brace enables the pivot axis to rotate about the axis of the thigh-receiving portion of the second brace by an angle required for hip correction. The first brace forces the calf to rotate by the aforementioned angle relative to the toe-forward position, so as to drive the thigh and femoral head to rotate simultaneously.

### CROSS-REFERENCE TO RELATED APPLICATIONS

This application is a continuation of International Application No. PCT/CN2023/124226, filed on Oct. 12, 2023, which claims priority to Chinese Patent Application No. 202222696141.4, filed on Oct. 13, 2022. The disclosures of the above-mentioned applications are hereby incorporated by reference in their entireties.

### TECHNICAL FIELD

The present disclosure relates to the field of hip joint correction or rehabilitation, and in particular to a brace device.

### BACKGROUND

Among children or adolescents, some people have abnormalities in their hip joints, which affect the patient's walking gait, such as toe-in gait or toe-out gait. Thus, a brace is needed to correct the movement of the leg in order to intervene in the development of the hip joint and gradually restore the patient to a normal walking posture. There still are other scenarios where the hip joint needs to be kept in a certain posture. For example, in the recovery period of Perthes disease (Avascular Necrosis of Femoral Head), or for patients with femoral head or femoral neck fractures, it is best to keep the femoral head of the affected limb in the hip acetabulum, or keep the femoral neck at a certain angle, both of which need to keep the posture of the leg at a certain angle.

The braces currently used to correct hip joint abnormalities, or the rehabilitation braces that maintain the hip joint posture, are only simple securing of the legs, and the correction and rehabilitation effects are poor.

### SUMMARY

A brace device is provided according to the present application to improve the correction or rehabilitation effect.

To achieve the above purpose, the brace device according to the present application includes:
a first brace for securing at least one side of a calf and a foot in a circumferential direction of the calf;
a second brace for accommodating at least one side of a thigh and secured relative to the body;
a pivotable connecting component, in which one end of the connecting component is fixedly connected to the first brace, and the other end of the connecting component is fixedly connected to the second brace, and a pivot axis of the connecting component coincides with a pivot axis of a knee joint when the brace device is put on; and
an installation position of the connecting component on the second brace makes the pivot axis rotate around the axis of the second brace and the thigh by an angle required for hip joint correction relative to a position of the connecting component when toes are facing straight forward.

In the embodiment of the present application, the first brace includes a calf brace and a foot brace, the calf brace is used to be mounted outside the calf, the foot brace is used to be mounted outside the foot, and the calf brace is fixedly connected to the foot brace so that the calf brace is relatively fixed to the calf in the circumferential direction of the calf.

In the embodiment of the present application, the second brace includes a thigh brace and a fixing device, the thigh brace is used to be mounted outside the thigh, and the fixing device is used to fix the thigh brace relative to the body.

In the embodiment of the present application, the fixing device includes a hip brace and a connecting mechanism, the hip brace is used to be mounted on the human hip and fixed relative to the body; and the connecting mechanism is used to connect the thigh brace to the hip brace.

In the embodiment of the present application, the connecting mechanism includes a first connecting rod and a movable joint, the movable joint is provided on the hip brace, one end of the first connecting rod is provided on the thigh brace, and the other end of the first connecting rod is provided on the movable joint; and
when the brace device is put on, the first connecting rod and the movable joint are located on left and right sides of the body, and are movably connected according to a preset degree of freedom, so that the thigh brace can move approximately in front and rear direction relative to the hip brace.

In the embodiment of the present application, the fixing device is a connecting member, the connecting member is used to connect the thigh braces of a left thigh and a right thigh on inner sides of legs, and the connecting member is rigidly connected to at least the thigh brace to be corrected.

In the embodiment of the present application, the connector includes a first connector and a second connector, one end of the first connector is connected to the thigh brace of the first side thigh, one end of the second connector is connected to the thigh brace of the second side thigh, and the other end of the first connector is connected to the other end of the second connector; and
an angle between the connector and the thigh brace determines an thigh abduction angle required for hip joint correction.

In the embodiment of the present application, the first connector and the second connector are both provided with multiple threaded holes in a length direction, and the first connector and the second connector select the threaded holes required for a total length of the connector from the multiple threaded holes for threaded connection.

In the embodiment of the present application, a fixed connection position of one end of the first connector and the second connector connected to each other in the length direction can be adjusted steplessly.

In the embodiment of the present application, the connector is connected to the thigh brace of the thigh on one side of the healthy hip joint through a universal joint.

In the embodiment of the present application, a thigh abduction angle can be adjusted by adjusting the angle between the connector and the thigh brace.

In an embodiment of the present application, the pivotable connecting assembly includes a first assembly, the first assembly includes a second connecting rod and a third connecting rod connected by a first pivot axis, the second connecting rod is fixedly connected to the calf brace, and the third connecting rod is fixedly connected to the thigh brace.

In an embodiment of the present application, the pivotable connecting assembly further includes a second assembly, the second assembly includes a fourth connecting rod and a fifth connecting rod connected by a second pivot axis, the fourth connecting rod is fixedly connected to the calf brace, the fifth connecting rod is fixedly connected to the thigh brace, the first assembly and the second assembly are respectively located on inner and outer sides of a corresponding leg, and axes of the first pivot axis and the second pivot axis coincide.

In an embodiment of the present application, positions of the third connecting rod and the fifth connecting rod in the circumferential direction of the thigh brace are adjustable.

In an embodiment of the present application, an arc-shaped slide rail coaxial with the thigh brace and arranged on an outer surface of the thigh brace is further included, and ends of the third connecting rod and the fifth connecting rod connected to the thigh brace are respectively provided with a first slider, and the first slider can be fixedly connected to the arc-shaped slide rail at an adjustable position selected by user.

In an embodiment of the present application, the pivoting connection assembly includes at least one hook and at least one hanging ring, the at least one hook is arranged along a circumference of the calf brace, and the at least one hanging ring is arranged along a circumference of the thigh brace; and the hook is fixedly connected to the thigh brace through the hanging ring.

In an embodiment of the present application, an axis of the calf brace is located on a plane where the second connecting rod and the fourth connecting rod are located; and
an axis of the thigh brace is located on a plane where the third connecting rod and the fifth connecting rod are located.

In an embodiment of the present application, the second connecting rod and the third connecting rod are telescopically connected to the calf brace in the length direction of the calf brace.

In an embodiment of the present application, the calf brace is provided with two slide grooves in the length direction, and the second connecting rod and the fourth connecting rod are slidably arranged in the two slide grooves.

In an embodiment of the present application, the second connecting rod and the fourth connecting rod are both provided with multiple through holes in the length direction, and the calf brace is provided with multiple threaded holes corresponding to the multiple through holes, and the second connecting rod and the fourth connecting rod are threadedly connected to the calf brace through the multiple through holes and the multiple threaded holes.

In the embodiment of the present application, the calf brace and the foot brace are integrally formed.

In the embodiment of the present application, the calf brace is provided with a first opening, and the first opening penetrates a side wall of the calf brace along the length direction of the calf brace;
the thigh brace is provided with a second opening, and the second opening penetrates the side wall of the thigh brace along the length direction of the thigh brace; and
both the first opening and the second opening are provided with straps.

The brace device according to the embodiments of the present application includes the first brace, the second brace and the pivotable connecting component, and the first brace fixes the calf and the foot in the circumferential direction of the calf, the second brace accommodates the thigh and is relatively fixed relative to the trunk, one end of the pivotable connecting component is fixedly connected to the first brace, and the other end of the pivotable connecting component is fixedly connected to the second brace. With the position of the pivot axis of the connecting component when the toes are facing straight ahead as a reference, the installation position of the connecting component on the second brace enables the pivot axis to rotate around the axis of the part of the second brace accommodating the thigh by an angle required for hip joint correction, so that the first brace can force the calf to rotate by the aforementioned angle relative to the toes-forward position, thereby driving the thigh and femoral head to rotate synchronously, and the second brace is generally fixed relative to the trunk, so that the first brace rotated relative to the second brace can force the foot, the calf, the thigh and the femoral head to rotate, that is, the femoral head can rotate by the said angle in the hip joint socket, thereby playing a role in hip joint correction or rehabilitation.

### BRIEF DESCRIPTION OF THE DRAWINGS

In order to more clearly illustrate the technical solutions in the embodiments of the present application or the prior art, the drawings required for use in the embodiments or the prior art descriptions are briefly introduced below. Obviously, the drawings described below are only some embodiments of the present application. For ordinary technicians in this field, other drawings can be obtained based on the structures shown in these drawings without creative work. In the drawings:

FIG. 1 is a schematic diagram of the structure of an embodiment of the brace device of the present application;

FIG. 2 is a schematic diagram of the structure of another embodiment of the brace device of the present application;

FIG. 3 is an enlarged view of a part B of the brace device in FIG. 2;

FIG. 4 is a schematic diagram of the structure of another embodiment of the brace device of the present application;

FIG. 5 is a schematic diagram of the structure of another embodiment of the brace device of the present application;

FIG. 6 is a schematic diagram of the structure of the brace device of the present application on a single left leg;

FIG. 7 is a schematic diagram of the structure of another embodiment of the brace device of the present application;

FIG. 8 is a schematic diagram of the structure of the right thigh brace and the right leg calf brace of the brace in FIG. 7;

FIG. 9 is a schematic diagram of the structure of the right thigh brace and the right leg calf brace of the brace in FIG. 4;

FIG. 10 is a schematic diagram of the structure of the right thigh brace and the right leg calf brace of the brace in FIG. 4 in another embodiment; and

FIG. 11 is an enlarged view of a part A of the brace device in FIG. 1.

### DETAILED DESCRIPTION OF THE EMBODIMENTS

The following will be combined with the drawings in the embodiments of the present application to clearly and completely describe the technical solutions in the embodiments of the present application. Obviously, the described embodiments are only part of the embodiments of the present application, not all of the embodiments. Based on the embodiments in the present application, all other embodiments obtained by ordinary technicians in this field without creative work are within the scope of protection of the present application.

It should be noted that if there are directional indications (such as up, down, left, right, front, back . . . ) in the embodiments of the present application, the directional indication is only used to explain the relative position relationship, movement, etc. between the components under a certain posture shown in a specific figure. If the specific posture changes, the directional indication will also change accordingly.

In addition, if there are descriptions of “first”, “second”, etc. in the embodiments of the present application, the descriptions of “first”, “second”, etc. are only used to distinguish the names of similar technical features, and cannot be understood as indicating or implying their relative importance or implicitly indicating the number of technical features indicated. Therefore, the features defined as “first” and “second” can explicitly or implicitly include at least one of the features. In addition, the technical solutions between the various embodiments can be combined with each other, but they must be based on the ability of ordinary technicians in this field to achieve them. When the combination of technical solutions is contradictory or cannot be achieved, it should be considered that such a combination of technical solutions does not exist and is not within the scope of protection required by this application.

The Applicant found that a main movement of a knee joint of a human leg is flexion and extension, and the flexion and extension movement belongs to a two-dimensional movement with only two degrees of freedom; in addition to flexion and extension, the knee joint can also help the human body achieve a limited angle of knee internal rotation or knee external rotation. However, due to the structural limitations of the knee joint itself, knee internal rotation or knee external rotation can only be achieved under the knee's bending state, and the angle is not large (the rotation range of knee internal rotation is 0°˜30°, and the rotation range of knee external rotation is 0°˜40°). At the same time, although the foot and the calf are not aligned in a straight line and the ankle joint is generally a joint that can move in all directions, the degree of freedom of each direction of movement is not large. Therefore, in general, the thigh, calf and foot can be kept on the same plane. Therefore, the rotation of the calf around the axis of the calf can drive the rotation of the entire leg around the axis of the leg, which means that the femoral head rotates inward or outward relative to the pelvis (that is, relative to the trunk) in the hip joint socket. For example, when the foot drives the leg to rotate inward along the axis, the femoral head corresponding to this leg rotates inward relative to the hip joint socket (referred to as “internal rotation”), and when the foot drives the leg to rotate outward along the axis, the femoral head corresponding to this leg rotates outward relative to the hip joint socket. When the leg remains in an inward rotation state, the corresponding femoral head remains in an internal rotation state, and when the leg remains in an outward rotation state, the corresponding femoral head remains in an external rotation state. Based on this, a brace device for correcting or rehabilitating the human hip joint is provided according to the present application.

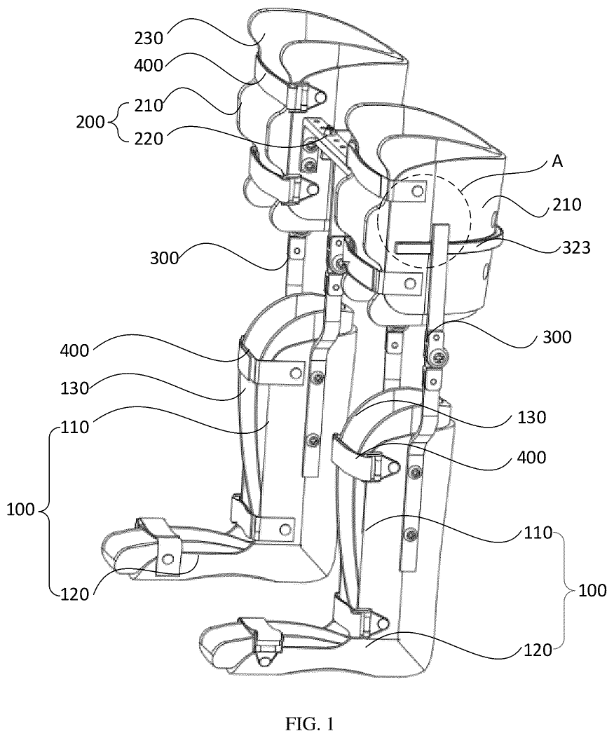

As shown in FIG. 1, the brace device provided by an embodiment of the present application includes a first brace 100 for securing at least one side of the calf and the foot in the circumferential direction of the calf; a second brace 200 for accommodating at least one side of the thigh and achieving relatively fixing relative to the body; a pivotable connecting component 300, one end of the connecting component 300 is fixedly connected to the first brace 100, and the other end of the connecting component 300 is fixedly connected to the second brace 200, and a pivot axis of the connecting component 300 coincides with the pivot axis of the knee joint when the first brace 100 is put on; an installation position of the connecting component 300 on the second brace 200 can make the pivot axis rotate around the axis of the second brace 200 and the thigh by an angle required for hip joint correction relative to a position of the connecting component 300 when toes are facing straight ahead.

The first brace 100 is used to fit the calf. When the first brace 100 is put on the calf of a human body, it can be fixed relatively to the leg in the circumferential direction of the leg under the fixing action of the foot. If the foot drives the first brace 100 to rotate inward, the leg can rotate inward accordingly; if the foot drives the first brace 100 to rotate outward, the leg can rotate outward accordingly; or vice versa, if the leg rotates inward, the foot drives the first brace 100 to rotate inward accordingly; if the leg rotates outward, the foot drives the first brace 100 to rotate outward accordingly. When the first brace 100 and the leg rotate inward or outward, the two remain relatively still. In addition, the first brace 100 can includes a left leg first brace 100 and a right leg first brace 100. The left leg first brace 100 and the right leg first brace 100 are mirror images of each other, and their functions and uses are completely the same. When the user uses it, if only the left hip joint needs to be corrected or rehabilitated, only the first brace 100 of the left leg needs to be put on; if only the right hip joint needs to be corrected or rehabilitated, only the first brace 100 of the right leg needs to be put on; if both the left and right hip joints need to be corrected or rehabilitated at the same time, both the left and right legs need to wear the corresponding first brace 100.

As shown in FIG. 1, in one embodiment of the present application, the first brace 100 on either side may include a calf brace 110 and a foot brace 120. The calf brace 110 is used to fit the calf, and the foot brace 120 is used to fit the foot, and the calf brace 110 is fixedly connected to the foot brace 120 so that the calf brace 110 is relatively fixed to the calf in the circumferential direction of the calf. In the embodiment of the present application, the calf brace 110 and the foot brace 120 used to fit the lower limb of the same side of the human body can be integrally formed, and then integrally fit the foot and calf of one side of the human body. In other embodiments, the calf brace 110 and the foot brace 120 for fitting any side of the lower limb of the human body can be formed separately, the calf brace 110 fits the calf part, and the foot brace 120 fits the foot part, and then the calf brace 110 and the foot brace 120 are fixedly connected, and the fixed connection position can fit the ankle joint of one side of the lower limb of the human body. Thus, when the foot drives the foot brace 120 and the calf brace 110 to rotate inward, the leg follows to rotate inward, and when the foot drives the foot brace 120 and the calf brace 110 to rotate outward, the leg follows to rotate outward; or vice versa, if the leg rotates inward, the foot drives the foot brace 120 and the calf brace 110 to rotate inward, and if the leg rotates outward, the foot drives the foot brace 120 and the calf brace 110 to rotate outward.

The second brace 200 is relatively fixed relative to the trunk. It should be noted that the second brace 200 in this application is relatively fixed relative to the trunk, and “relative fixation” does not mean absolute fixation. Due to the flexibility of the human body, the human body can perform some subtle movements within a small range. When the body performs some subtle movements, a slight displacement may occur between the second brace and the trunk; in addition, the fixing method of the second brace 200 in this application and the trunk itself needs to provide a certain degree of activity for the thigh, such as a certain degree of walking movement, sitting on the ground, bending left and right, front and back, sitting, etc., The second brace 200 in this application is relatively fixed relative to the trunk, but in the most common postures of the human body (such as normal standing, normal sitting), the second brace 200 can keep the thigh position relative to the trunk at or very close to the position required for correction. In other words, in the context of this specification, the essence of the second brace 200 being relatively fixed relative to the trunk is that the second brace 200 can keep the thigh roughly at the position required for correction relative to the hip bone.

Therefore, the second brace 200 can also provide a reference for the rotation of the first brace 100 in the circumferential direction of the leg. Because the position of the second brace 200 relative to the hip joint (and/or the body) is generally fixed, the first brace 100 and the calf are rotated by the angle relative to the second brace 200 when the first brace 100 and the calf rotate a certain angle relative to the leg axis.

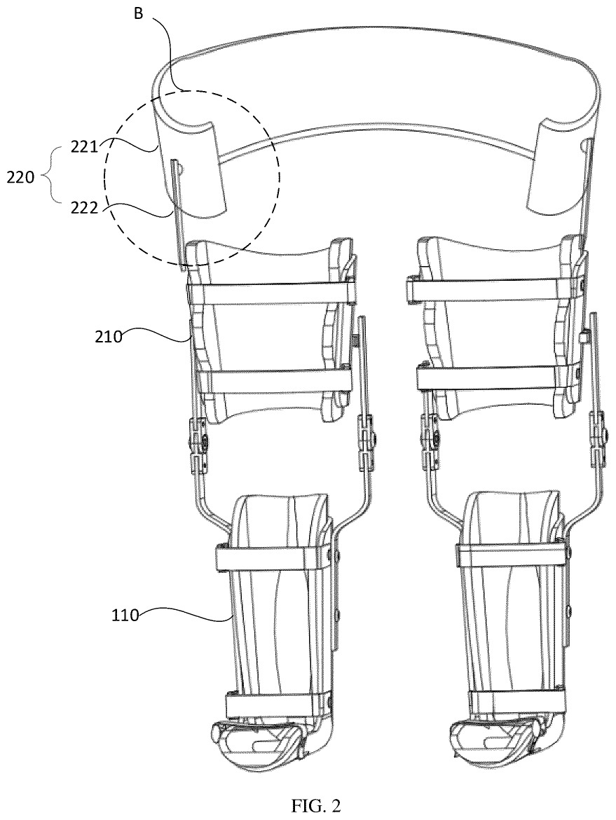

There are many ways to fix the second brace 200 relative to the hip joint and/or the body. For example, in one embodiment, as shown in FIG. 2, the second brace 200 includes a thigh brace 210 and a fixing device 220, a thigh brace 210 is used to fit the thigh of the lower limb of the human body, and the fixing device 220 is used to fix the thigh brace 210 relative to the body. Similarly, the fixing device 220 described in the present application is used to fix the thigh brace 210 relative to the body, which is not an absolute fixation, because the body has flexibility, and the thigh brace is only fixed relative to the body. For example, the fixing device 220 is fixed in the circumferential direction of the human body, especially the hip joint, so that the thigh brace 210 is fixed relative to the human body, especially the hip joint. For example, in the embodiment of the present application, the fixing device 220 can be a hip brace 221 and a connecting mechanism 222 fixed around the hip joint. The form of the hip brace 221 can be similar to the thigh brace 210, or it can be a frame structure. The connecting mechanism 222 can fix the thigh brace 210 to the hip brace 221 in the circumferential direction of the body. Then, when the calf brace 110 is rotated by a certain angle and connected to the thigh brace 210, the first brace 100 (including the calf brace 110) can be kept fixed, and then the leg in the first brace 100 is kept in an external rotation state or an internal rotation state since the thigh brace 210 is fixed to the hip brace 221 in the circumferential direction of the body, that is, the thigh brace 210 cannot rotate along the axis of the human body.

In this embodiment, however, if the connection mechanism 222 between the thigh brace 210 and the hip brace 221 is in a hard connection, the leg, hip joint and trunk of the patient wearing the brace device can only maintain a fixed posture, and cannot move forward and backward, such as lying or sitting. To solve this problem, the connection mechanism 222 connecting the hip brace 221 and the thigh brace 210 can be set to a two-dimensional (planar) motion structure that is roughly consistent with the direction of movement of the knee joint (for example, connecting the two braces by a rotational connection), or set to an omnidirectional motion structure with a certain limit on the range of motion as needed (for example, connecting the two braces by a universal swivel joint).

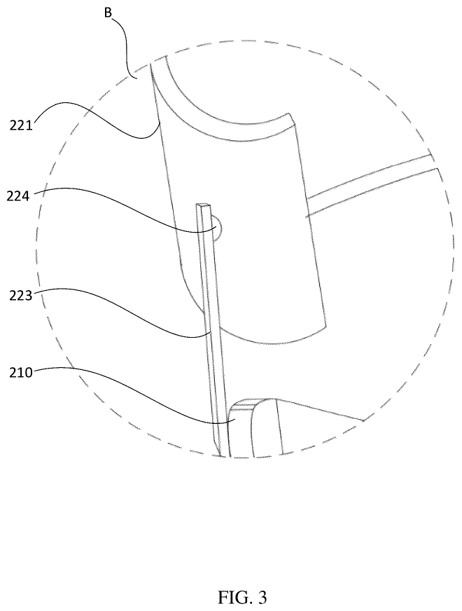

As shown in FIG. 3, in one embodiment of the present application, the connecting mechanism 222 includes a first connecting rod 223 and an active joint 224, and the active joint 224 is disposed on the hip brace 221, one end of the first connecting rod 223 is disposed on the thigh brace 210, and the other end of the first connecting rod 223 is disposed on the active joint 224; when the brace device is put on, the first connecting rod 223 and the active joint 224 are located on the left and right sides of the body, and are movably connected at the front and back sides of the body according to a preset degree of freedom, so that the thigh brace 210 can move approximately forward and backward relative to the hip brace 221.

The first connecting rod 223 and the active joint 224 can be both disposed on the outside of the leg, and the active joint 224 is, for example, a universal joint, and the active angle of the active joint 224 is limited to the movement at the front and back sides of the body according to a preset degree of freedom, while the movement around the body needs to be restricted to prevent the thigh brace 210 from rotating relative to the body.

In another embodiment, the first connecting rod 223 and the movable joint 224 are movably connected at the left and right sides of the trunk according to the preset degrees of freedom. It should be noted that the left and right sides here refer to the direction of the legs abducting to the left and right sides of the trunk. The first connecting rod 223 and the movable joint 224 are arranged at the left and right sides of the trunk to allow the legs to abduct to the left and right sides of the trunk.

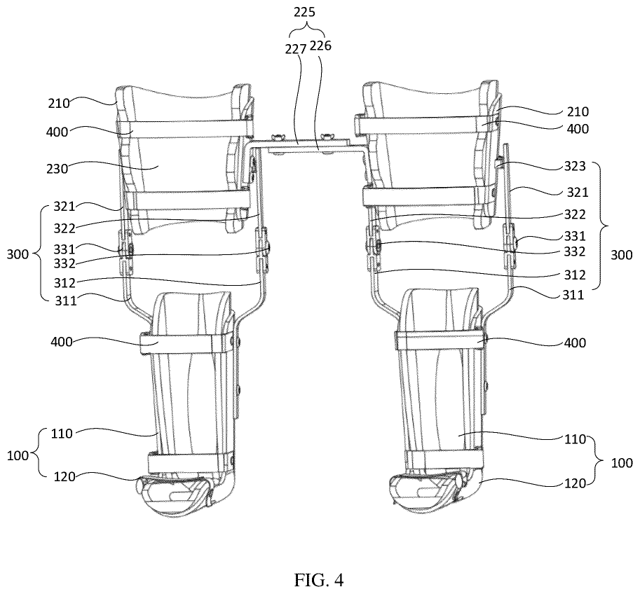

As shown in FIGS. 1 and 4, in one embodiment of the present application, the fixing device is a connecting member 225, and the connecting member 225 connects the thigh brace 210 of the left thigh and the right thigh on the inner side of the leg, and the connecting member 225 is rigidly connected to at least the thigh brace 210 on the side to be corrected.

As shown in FIGS. 1 and 4, in another embodiment, the two thigh braces 210 of the left and right legs connected to each other by the connecting member 225 together constitute a mechanism for maintaining relative fixation in the circumferential direction relative to the trunk (hip joint). The rigid connection between the connector 225 and the thigh brace 210 on the “to be corrected” side, and the position of the thigh brace 210 on the healthy side in the general position state can force the thigh on the side to be corrected to be in the desired position relative to the whole body (the angle between the connector 225 and the thigh brace 210 and the length of the connector 225 determine the thigh outward angle and the position relative to the hip bone). If both hip joints need to be corrected at the same time, the connector 225 is rigidly connected to the thigh braces 210 on both sides, and their positions are relatively fixed due to the constraints on the other side. Although the legs can move relative to the body and hip joints, whether alone or together, when the legs are connected in a relatively fixed position, their range of motion relative to the hip joint is limited. Moreover, when the human body is in a normal posture, the plane formed by the legs is generally consistent with the plane where the body and hip joints are located, or is consistent in statistical average.

Therefore, when the whole of the two legs is used as the reference position, the internal rotation or external rotation formed by the first brace 100 can maintain the internal rotation or external rotation of the leg relative to the hip joint or the body in most of the time, or in a statistical average sense, so as to play a correction or rehabilitation role. It should be noted that in this embodiment, even if only the left leg needs to be corrected, the right leg still needs to have a thigh brace 210 (but since the right leg does not need to be corrected, the first brace 100 for the right leg may not be needed, or it may be set in a normal posture) so as to form a fixed structure together with the thigh brace 210 of the left leg. At this time, the connecting member 225 can only be rigidly connected to the thigh brace 210 of the left leg (the thigh to be corrected), and does not need to be rigidly connected to the thigh brace of the right leg (the thigh that does not need to be corrected). When both the left and right hip joints need to be corrected, the connecting member 225 needs to be rigidly connected to both the thigh brace 210 of the left leg and the thigh brace 210 of the right leg.

In one embodiment of the present application, a connector 225 may be provided between the thigh braces 210 of the left and right thighs, and the thigh brace 210 of the left thigh and the thigh brace 210 of the right thigh are connected by the connector 225, so that the thigh brace 210 of the left thigh and the thigh brace 210 of the right thigh are combined to form a fixed structure in the circumferential direction of the body.

The distance between the legs of users for different ages and heights may be different, so the length of the connector 225 can be set according to the distance between the left and right thighs of the user. In addition, a connector 225 whose length can be adjusted telescopically can also be provided to improve versatility. For example, in another embodiment of the present application, the connector 225 may include a first connector 226 and a second connector 227, one end of the first connector 226 is connected to the thigh brace 210 of the first thigh, one end of the second connector 227 is connected to the thigh brace 210 of the second thigh, the other end of the first connector 226 is connected to the other end of the second connector 227, and the angle between the connector 225 and the thigh brace 210 determines the thigh abduction angle required for hip joint correction.

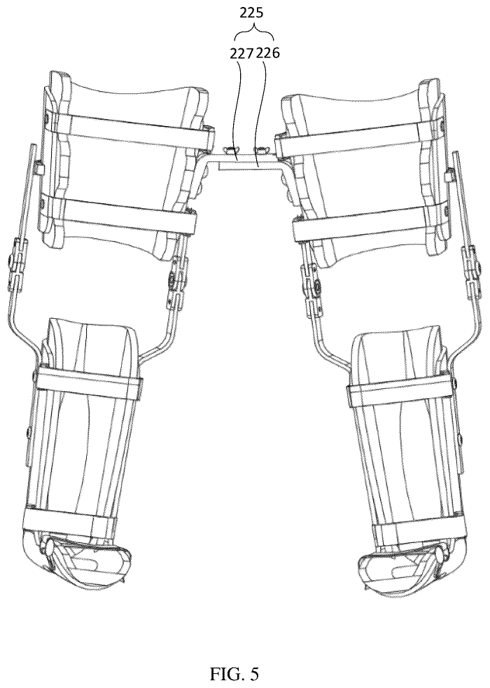

As shown in FIG. 5, in the embodiment of the present application, the first connector 226 and the second connector 227 are both provided with multiple threaded holes in the length direction, and the first connector 226 and the second connector 227 can select the threaded holes required for the total length of the connector 225 from the threaded holes for threaded connection. For example, the first connector 226 and the second connector 227 are both provided with multiple threaded holes in the length direction, so that when the first connector 226 and the second connector 227 are connected, appropriate threaded holes can be selected for threaded connection according to the total length required for the connector 226.

In another embodiment of the present application, the fixed connection position of one end of the first connecting member 226 and the second connecting member 227 connected to each other in the length direction can be adjusted steplessly.

For example, in other embodiments, the first connecting member 226 and the second connecting member 227 can also be set to a sliding connection, such as setting a slide rail on the first connecting member 226, and setting the second connecting member 227 on the slide rail, and the distance between the left and right thighs can be adjusted by sliding. For another example, the first connecting member 226 can also be set to be hollow, and the first connecting member 226 is mounted on the outside of the second connecting member 227, so as to form a sliding connection. It should be noted that when the first connecting member 226 and the second connecting member 227 are slidably connected, when the two slide relative to each other to a suitable length position, they are still fixedly connected, that is, the fixed connection position of the first connecting member 226 and the second connecting member 227 in the length direction can be adjusted steplessly.

Through the above-mentioned threaded connection or sliding connection, the purpose of adjusting the distance between the left and right thighs can be achieved. In addition to adapt to users of different heights, weights, and ages, this form can also achieve the purpose of abduction correction by adjusting the length of the connecting member 225 when the user's hip joint needs to be corrected or rehabilitated.

For example, if the user's left and right hip joints need to be corrected outward, the length of the connecting member 225 can be appropriately increased at this time, so that the thigh brace 210 of the left leg and the thigh brace 210 of the right leg are both extended outward. When the user wears it, the left leg and the right leg are both extended outward compared to the normal state, so that the left leg drives the left hip joint to expand outward, and the right leg drives the right hip joint to expand outward, thereby achieving the purpose of correction or rehabilitation of the hip joint to expand outward.

In addition, as shown in FIG. 5, the first connecting member 226 and one end of the second connecting member 227 connected to the thigh brace 210 can be set with an appropriate bending angle to cooperate with the abduction correction of the legs. The bending angle of the first connecting member 226 and one end of the second connecting member 227 connected to the thigh brace 210 can be set according to the thigh abduction angle required for hip joint correction, and different bending angles can be set according to different abduction angles. The thigh abduction angle required for hip joint correction can be determined by the angle between the connecting member 225 and the two thigh braces 210. The connecting member 225 and the two thigh braces located above the connecting member 225 roughly form an isosceles triangle structure, the two thigh braces 210 are the two waists of the isosceles triangle, and the connecting member 225 is the base of the isosceles triangle. When the thigh abduction angle required for hip joint correction is determined, that is, the vertex angle of the isosceles triangle is known, then the base angle of the isosceles triangle can be calculated, so that the angle between the connecting member 225 and the thigh brace 210 is set to the base angle of the corresponding size, which can ensure that the two thigh braces 210 can provide the thigh abduction angle required for hip joint correction.

In another embodiment, the thigh abduction angle can be adjusted by adjusting the angle between the connecting member 225 and the thigh brace 210. For example, the first connector 226 and the second connector 227 can be connected to the thigh brace 210 in a hinged manner, so that the distance between the left and right thigh braces 210 can be adjusted by the first connector 226 and the second connector 227 according to the required abduction angle, and the first connector 226 and the second connector 227 are both hinged to the thigh brace 210, so when the distance between the first connector 226 and the second connector 227 is adjusted, the angle between the thigh brace 210 and the first connector 226 and the second connector 227 can be adjusted accordingly.

As shown in FIG. 5, in another embodiment of the present application, the connector 225 is connected to the thigh brace 210 of the thigh on one side of the healthy hip joint through a universal joint. For example, assuming that only the left hip joint needs to be corrected or rehabilitated, the connector 225 and the thigh brace 210 of the right thigh can be connected through a universal joint, so that the mobility of the right thigh on the healthy side can be increased, and the position of the left thigh and the entire hip that needs to be corrected can be fixed by the right healthy thigh.

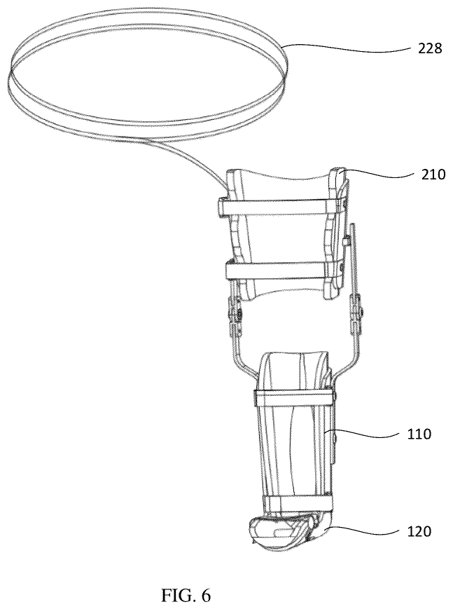

In another embodiment, as shown in FIG. 6, the fixing device 220 can also be a fixing belt 228. Taking the left thigh brace 210 as an example, when the first brace 100 is put on, the left thigh brace 210 is placed on the outer periphery of the left thigh, and then the fixing belt 228 is fixed to the waist or the body. For example, the fixing belt 228 can be wrapped around the waist or the body for several turns to form a fixed connection, so that the fixing belt 228 fixed in the circumference of the body pulls the thigh brace 210 and make it not rotate in the circumference of the left thigh.

As shown in FIG. 4, in the embodiment of the present application, the connecting assembly 300 is used to connect the first brace 100 and the second brace 200, and the second brace 200 has at least one connecting station in the circumference of the part for accommodating the thigh. For example, in one embodiment, only one connecting station is provided in the circumferential direction of the part of the second brace 200 for accommodating the thigh, and a mechanism is formed by the connecting station to fix the first brace 100 relative to the second brace 200 (for example, the fixing plane associated with the hip joint or the body in the aforementioned embodiment, or the fixing plane formed by the second braces 200 on the left and right sides) in the circumferential direction to a desired angle, such as inward rotation of 5° or 15°, etc. (the specific angle value depends on the needs of correction or rehabilitation, and can be determined according to actual conditions. In the case of only one connecting station, since the degree is not adjustable, the entire brace needs to be customized according to the correction or rehabilitation plan), then when the user wears the first brace 100, the first brace 100 can drive the leg to rotate inwardly by 5° or 15°, etc., and then the first brace 100 is fixedly connected to the second brace 200 through the connecting assembly 300 through this connecting station, so that the user's femoral head can be rotated inwardly relative to the hip joint according to the correction range of 5° or 15°. It should be noted that when the first brace is rotated inward by 5° or 15°, the connecting component is also rotated inward by 5° or 15°, that is, the axis of the connecting component rotates around the axis of the part of the second brace for accommodating the thigh by an angle, and this angle is the angle required for correction or rehabilitation, and is not limited to a fixed angle.

In other embodiments, the second brace 200 can also be provided with multiple connection stations. For example, a station where the first brace 100 drives the leg to rotate inward by 10° and then connects with the second brace 200, or a station where the first brace 100 drives the leg to rotate outward by 10° and then connects with the second brace 200. The specific number of the multiple connection stations in this embodiment is not limited, and can be multiple connection stations with fixed correction angles, or can be connection stations with any correction angle, that is, whether the first brace 100 is rotated inward by a certain angle or rotated outward by a certain angle, the second brace 200 can provide corresponding connection stations for connecting the first brace 100 and the second brace 200. In this case, the correction or rehabilitation brace device of this embodiment is more universal. Any user can select the appropriate connection position according to the correction or rehabilitation program process, without having to customize a brace device of a specific angle.

As shown in FIG. 6, in the embodiment of the present application, the pivotable connection component 300 includes a first component, which includes a second connection rod 311 and a third connection rod 321 connected by a first pivot shaft 331, the second connection rod 311 is fixedly connected to the calf brace 110, and the third connection rod 321 is fixedly connected to the thigh brace 210.

As shown in FIG. 3, in the embodiment of the present application, the first component may include the second connecting rod 311, which is fixedly connected to the calf brace 110 in the circumferential direction of the calf brace 110, and the second connecting rod 311 is arranged parallel to the axis of the calf brace 110; the second component includes a third connecting rod 321, which is fixedly connected to the thigh brace 210 in the circumferential direction of the thigh brace 210, and the third connecting rod 321 is arranged parallel to the axis of the thigh brace 210; the second connecting rod 311 and the third connecting rod 321 are located on the same side of the leg, and after the calf brace 110 is rotated by a preset angle, the second connecting rod 311 and the third connecting rod 321 are connected.

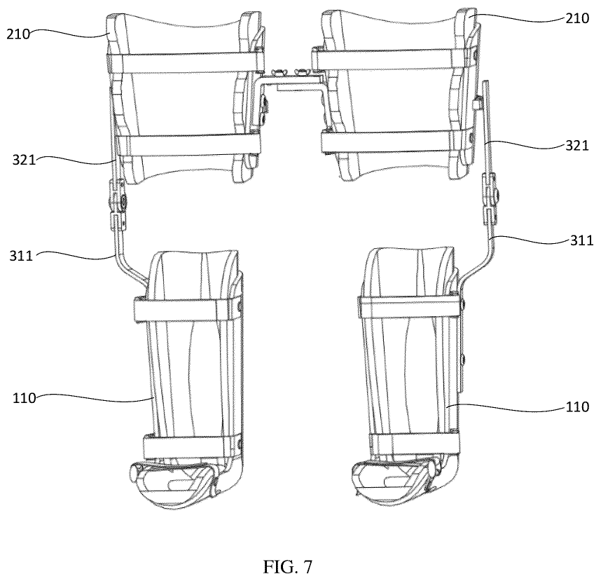

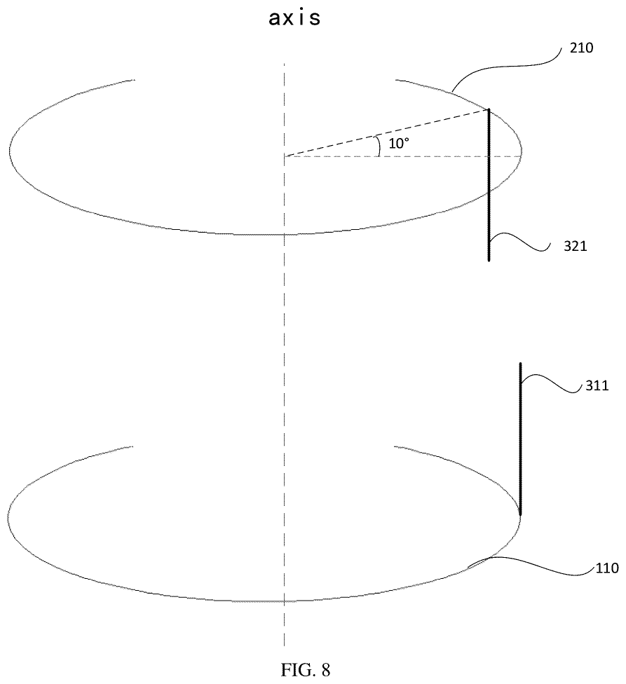

The second connecting rod 311 and the third connecting rod 321 can both be of a long connecting rod, and the second connecting rod 311 and the third connecting rod 321 are both located at the inner side of the leg, or at the outer side of the leg. As shown in FIG. 8, FIG. 8 is a schematic diagram of the connection between the thigh brace 210 and the calf brace 110 of the right leg in FIG. 7, and the second connecting rod 311 and the third connecting rod 321 are both located on the outer side of the leg, and the third connecting rod 321 is located at a position 10° rotated inwardly on the left side of the thigh brace 210, and “10°” is the correction or rehabilitation angle, and the second connecting rod 311 is located on the right side of the calf brace 110. Therefore, when the calf brace 110 is connected to the thigh brace 210, the calf brace 110 needs to be rotated 10° inwardly so that the second connecting rod 311 can be connected to the third connecting rod. After the connection, the calf brace 110 and the foot brace 120 connected to the calf brace 110 have been rotated 10° inwardly. When the user uses this brace device, the right foot can rotate 10° inwardly, and the right femoral head can also rotate 10° inwardly synchronously. When the user's right leg wears this brace device, the right calf and the right foot are supported by the right calf brace 110 and the foot brace 120. The right leg brace 120 is limited, and the right calf brace 110 is connected by the second connecting rod 311 and the third connecting rod 321. Therefore, the thigh brace 210 continuously provides the traction force of inward rotation to the calf brace 110 through the second connecting rod 311 and the third connecting rod 321, so that the right foot can continue to be in a state of 10° inward rotation, which can also make the femoral head of the user's right leg rotate inward relative to the hip joint according to the correction amplitude of 10° rotation to the inner side of the leg, thereby correcting or rehabilitating the right hip joint by 10° inward rotation. It should be noted that in the embodiment of the present application, the right hip joint needs to be rotated inwardly for correction or rehabilitation by 10°, so the third connecting rod 321 is set at a position rotated 10° inward from the right side of the thigh brace 210.

In other embodiments, it can also be correction or rehabilitation of other rotation amplitudes, such as 5° or 15°, and it can also be correction or rehabilitation of rotation to the outside. Whether it is correction or rehabilitation of rotation to the inside or correction or rehabilitation to the outside, the third connecting rod 321 can be set at the position where the outer wall of the thigh brace 210 rotates inwardly or outwardly by a corresponding angle, so that the calf brace 110 needs to be rotated inwardly or outwardly by the preset angle before it can be connected through the second connecting rod 311 and the third connecting rod 321.

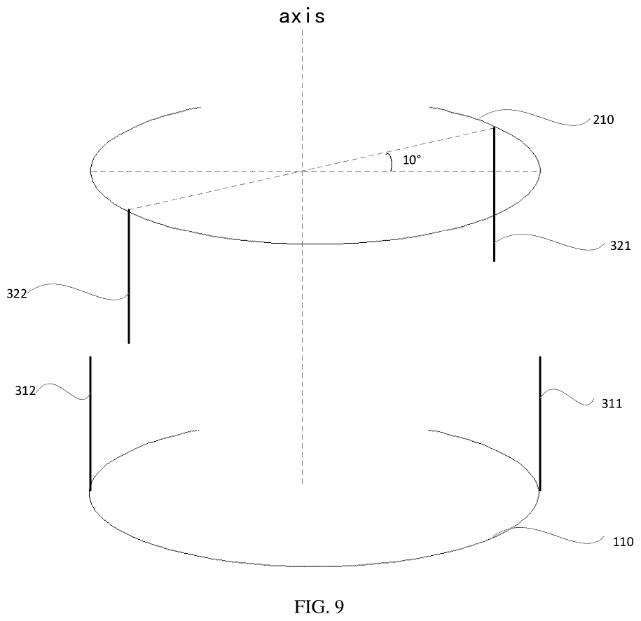

As shown in FIG. 4, in the embodiment of the present application, the pivotable connection component 300 also includes a second component, which includes a fourth connecting rod 312 and a fifth connecting rod 322 connected by a second pivot shaft 332, the fourth connecting rod 312 is fixedly connected to the calf brace 110, and the fifth connecting rod 322 is fixedly connected to the thigh brace 210. The first component and the second component are respectively located on inner and outer sides of the corresponding leg, and the axes of the first pivot shaft 331 and the second pivot shaft 332 coincide. The fourth connecting rod 312 is arranged in parallel with the second connecting rod 311 in the circumferential direction of the calf brace 110, and the second connecting rod 311 and the fourth connecting rod 312 are respectively located at inner and outer sides of the calf brace 110; the fifth connecting rod 322 is arranged in parallel with the third connecting rod 321 in the circumferential direction of the thigh brace 210, and the third connecting rod 321 and the fifth connecting rod 322 are respectively located at inner and outer sides of the calf; after the calf brace 110 is rotated by a preset angle, the second connecting rod 311 is connected to the third connecting rod 321, and the fourth connecting rod 312 is connected to the fifth connecting rod 322. As shown in FIG. 9, FIG. 9 is a schematic diagram of the calf brace 110 and the thigh brace 210 on the right leg in FIG. 4. In the embodiment of the present application, the second connecting rod 311 and the third connecting rod 321 are respectively located on the outside of the calf brace 110 and the thigh brace 210, and the fourth connecting rod 312 and the fifth connecting rod 322 are respectively located on the inside of the calf brace 110 and the thigh brace 210. Still taking the example that the right hip joint needs to be corrected by 10° inward rotation, the third connecting rod 321 is located at the position of the thigh brace 210 rotated 10° inwardly on the right side, the fifth connecting rod 322 is located at the position of the thigh brace 210 rotated 10 degrees from the back to the left side, the second connecting rod 311 is located at the position of the calf brace 110 on the right side, and the fourth brace is located at the position of the calf brace 110 on the left side. Therefore, when the calf brace 110 is connected to the thigh brace 210, the calf brace 110 needs to be rotated 10° inwardly so that the second connecting rod 311 can be connected to the third connecting rod 321, and the fourth connecting rod 312 can be connected to the fifth connecting rod 322. After the connection, the calf brace 110 and the foot brace 120 connected to the calf brace 110 have been rotated 10° inwardly. Then when the user uses this brace device, the right leg will rotate 10° inwardly, and the right femoral head will rotate 10° inwardly synchronously. When the leg wears this brace, the right foot is restricted by the right foot brace 120, and the right foot brace 120 is fixedly connected to the calf brace 110, and the thigh brace 210 continuously provides traction to the inside of the leg to the second connecting rod 311 and the fourth connecting rod 312 through the third connecting rod 321 and the fifth connecting rod connecting rod, so that the right calf and foot will continue to be in a state of inward rotation, so that the user's right femoral head can be in a state of inward rotation relative to the right hip joint according to the correction amplitude of 10°, thereby playing a correction or rehabilitation effect of 10° internal rotation on the right hip joint.

In one embodiment of the present application, a connecting rod can be set on both the inner and outer sides of the calf brace 110 and the thigh brace 210. During the correction or rehabilitation process, a traction force is applied onto the calf brace 110 from the inner and outer sides of the leg respectively. On the one hand, the force is more evenly distributed, and on the other hand, a firmer traction force can be applied, so as to prevent the calf brace 110 from rotating around the body.

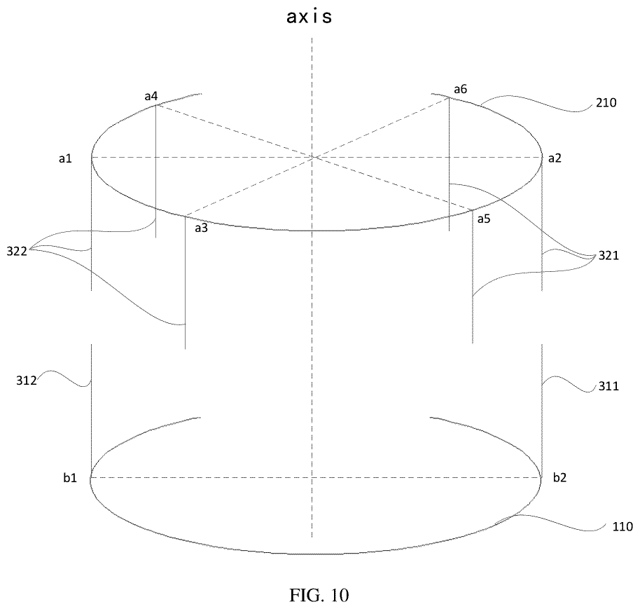

In the embodiment of the present application, the third connecting rod 321 and the fifth connecting rod 322 are adjustable in the circumferential direction of the thigh brace 210. As shown in FIG. 10, FIG. 10 is a schematic diagram of the structure of the right thigh brace 210 and the right calf brace 110, the left side is the inner side of the leg, the right side is the outer side of the leg, a1 and a2 respectively represent the left side and the right side of the thigh brace 210, and b1 and b2 respectively represent the left side and the right side of the calf brace 110. When the third connecting rod 321 is at position a2, the fifth connecting rod 322 is at position a1, when the third connecting rod 321 is at position a6, the fifth connecting rod 322 is at position a3, when the third connecting rod 321 is at position a5, the fifth connecting rod 322 is at position a4, that is, the third connecting rod 321 can be adjusted to the area on the right side of the thigh brace 210 to the inner side of the leg, and can also be adjusted to the area on the right side of the thigh brace 210 to the outer side of the leg. Similarly, the fifth connecting rod 322 can be adjusted to the area on the left side of the thigh brace 210 to the inner side of the leg, and can also be adjusted to the area on the left side of the thigh brace 210 to the outer side of the leg. As for the specific positions of the third connecting rod 321 and the fifth connecting rod 322, they can be determined according to the correction direction and correction angle. It should be noted that the third connecting rod 321 and the fifth connecting rod 322 are adjusted synchronously during the adjustment process.

For example, for the right leg, when the hip joint of the right leg needs to be corrected or rehabilitated inwardly, the fifth connecting rod 322 can be set at the a3 position, and the third connecting rod 321 can be set at the a6 position, so that the fourth connecting rod 312 at the b1 position and the second connecting rod 311 at the b2 position on the calf brace 110 need to be rotated inwardly by a certain angle before they can be fixedly connected with the third connecting rod 321 and the fifth connecting rod 322 in the circumferential direction of the leg. When the hip joint of the right leg needs to be corrected or rehabilitated outwardly, the fifth connecting rod 322 can be set at the a4 position, and the third connecting rod 321 can be set at the a5 position, so that the calf brace 110 needs to be rotated outwardly by a certain angle before the second connecting rod 311 and the fourth connecting rod 312 can be fixedly connected with the third connecting rod 321 and the fifth connecting rod 322 in the circumferential direction of the leg.

When in use, the third connecting rod 321 and the fifth connecting rod 322 first are adjusted to the corresponding positions, then the calf brace 110 is rotated to the corresponding angle and connected to the thigh brace 210, and then the user can wear the brace device for correction or rehabilitation.

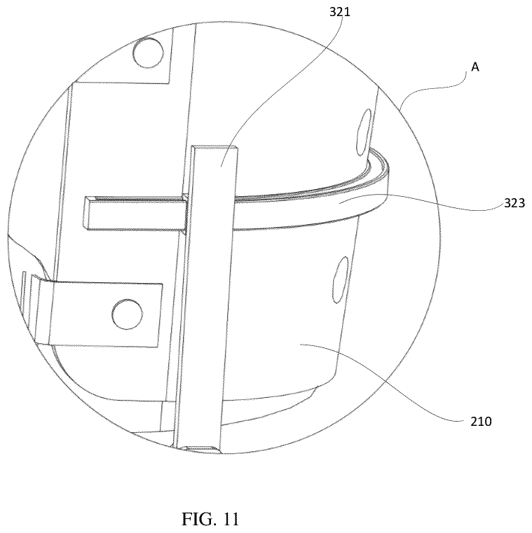

As shown in FIGS. 1, 4, and 11, in the embodiment of the present application, an arc-shaped slide rail 323 coaxial with the thigh brace 210 and arranged on the outer surface of the thigh brace 210 is further provided, and ends of the third connecting rod 321 and the fifth connecting rod 322 connected to the thigh brace 210 are respectively provided with a first slider, and the first slider can be fixedly connected to the arc-shaped slide rail 323 at a user-selected, adjustable position. FIG. 11 is a partial enlarged view of the area A in FIG. 1, the arc-shaped slide rail 323 is mounted on the outside of the thigh brace 210, the third connecting rod 321 and the fifth connecting rod 322 are both provided with the first slider, and the third connecting rod 321 and the fifth connecting rod 322 are both arranged in the arc-shaped slide rail 323 through the first slider, so that the third connecting rod 321 and the fifth connecting rod 322 can slide in the arc-shaped slide rail 323 through the first slider. It should be noted that after the third connecting rod 321 and the fifth connecting rod 322 slide to a suitable position in the arc-shaped slide rail 323, they still can be fixedly connected to the arc-shaped slide rail 323. Then, when the hip joint needs to be corrected or rehabilitated toward the inside of the leg, the third connecting rod 321 and the fifth connecting rod 322 can slide along the slide groove toward the inside of the leg to a suitable position and then be fixed. Then, the calf brace 110 is rotated inwardly by a corresponding angle, and then connected to the third connecting rod 321 and the fifth connecting rod 322 respectively through the second connecting rod 311 and the fourth connecting rod 312, and then the brace device can be put on by the user; when the hip joint needs to be corrected or rehabilitated toward the outside of the leg, the third connecting rod 321 and the fifth connecting rod 322 can slide along the arc-shaped slide rail 323 toward the outside of the leg to a suitable position and then be fixed. Then, the calf brace 110 is rotated outwardly by a corresponding angle, and then connected to the third connecting rod 321 and the fifth connecting rod 322 respectively through the second connecting rod 311 and the fourth connecting rod 312, and then the brace device can be put on by the user. By setting the arc-shaped slide rail 323, whether correction is required to be made to the inside or outside, and any correction angle can be set by the arc-shaped slide rail 323, thereby improving versatility.

As shown in FIG. 1 and FIG. 4, in the embodiment of the present application, the second connecting rod 311 and the third connecting rod 321 are rotationally connected in the bending direction of the knee joint, and the fourth connecting rod 312 and the fifth connecting rod 322 are rotationally connected in the bending direction of the knee joint. The fifth connecting rod 322 and the third connecting rod 321 are set on the inner and outer sides of the thigh brace 210, respectively, and the fourth connecting rod 312 and the second connecting rod 311 are set on the inner and outer sides of the calf brace 110, respectively, thus, when the second connecting rod 311 and the fourth connecting rod 312 are connected to the third connecting rod 321 and the fifth connecting rod 322, respectively, in the circumferential direction of the leg, each connecting rod forms a fixed connection with the thigh brace 210 and the calf brace 110, thereby ensuring the calf brace 110 not to rotate around the calf at an excessive angle, which may otherwise damage the user's leg or knee. In the embodiment of the present application, the second connecting rod 311 and the third connecting rod 321 can also be set to be rotationally connected in the bending direction of the knee joint, and the fourth connecting rod 312 and the fifth connecting rod 322 can be set to be rotationally connected in the bending direction of the knee joint. For example, a first pivot shaft 331 is set at one end where the second connecting rod 311 and the third connecting rod 321 are connected, and a second pivot shaft 332 is set at one end where the fourth connecting rod 312 and the fifth connecting rod 322 are connected. The first pivot shaft 331 makes the second connecting rod 311 and the third connecting rod 321 form a rotational connection in the bending direction of the knee joint, and the second pivot shaft 332 makes the fourth connecting rod 312 and the fifth connecting rod 322 form a rotational connection in the bending direction of the knee joint. Then, after the user wears the brace device of this embodiment, the bending function of the knee cannot be affected.

As shown in FIGS. 9 and 10, in the embodiment of the present application, the axis of the calf brace 110 is located on the plane where the second connecting rod 311 and the fourth connecting rod 312 are located; the axis of the thigh brace 210 is located on the plane where the third connecting rod 321 and the fifth connecting rod 322 are located. The axes of the second connecting rod 311, the fourth connecting rod 312 and the calf brace 110 are arranged in the same plane, and the axes of the third connecting rod 321, the fifth connecting rod 322 and the thigh brace 210 are arranged in the same plane. Then, in the wearing state, the second connecting rod 311, the fourth connecting rod 312, the axis of the user's calf, and the third connecting rod 321, the fifth connecting rod 322, the axis of the user's thigh are all located in the same plane, and the second connecting rod 311 and the third connecting rod 321, as well as the fourth connecting rod 312 and the fifth connecting rod 322, respectively provide traction to the calf brace 110 from both sides of the symmetrical leg axis, so that the force on the calf brace 110 is more uniform, and a better correction effect is achieved.

In the embodiment of the present application, the second connecting rod 311 and the fourth connecting rod 312 are telescopically connected to the calf brace 110 in the length direction of the calf brace 110. Therefore, when the brace device is put on, the second connecting rod 311 and the fourth connecting rod 312 can be adjusted by telescopically extending the length of the calf brace 110 to adapt to users of different heights, weights, and ages. Not only can it meet the needs of people of different heights/leg lengths, but users can also make adaptive adjustments during the correction process as their height changes with age.

There are many ways to adjust the length of the second connecting rod 311 and the fourth connecting rod 312 extending out of the calf brace 110. For example, as shown in FIG. 1, in the embodiment of the present application, the second connecting rod 311 and the fourth connecting rod 312 are both provided with multiple perforations in the length direction, and the calf brace 110 is provided with multiple threaded holes corresponding to the multiple perforations, and the second connecting rod 311 and the fourth connecting rod 312 are threadedly connected to the calf brace 110 through the multiple perforations and the multiple threaded holes. Multiple threaded holes are arranged and spaced apart on both the inner and outer sides of the calf brace 110, and the multiple threaded holes on each side are arranged along the length direction of the calf brace 110. The second connecting rod 311 and the fourth connecting rod 312 are also provided with multiple perforations in their length direction, and the second connecting rod 311 and the fourth connecting rod 312 can be fixed to the inner and outer sides of the calf brace 110 by screws. When the position needs to be adjusted, the screws are first loosened, the second connecting rod 311 and the fourth connecting rod 312 are adjusted to a suitable position, and then the screws are tightened. It should be noted that in other embodiments, the third connecting rod 321 and the fifth connecting rod 322 on the thigh brace 210 can also be similarly arranged, so the third connecting rod 321 and the fifth connecting rod 322 can also be adjusted in the length direction of the thigh brace 210 to change the length of the third connecting rod 321 and the fifth connecting rod 322 extending out of the thigh brace 210.

For example, in another embodiment, the calf brace 110 is provided with two slide grooves in the length direction, and the second connecting rod 311 and the fourth connecting rod 312 are slidably arranged in the two slide grooves. The two slide grooves are respectively located at the inner and outer sides of the calf brace 110 corresponding to the second connecting rod 311 and the fourth connecting rod 312. Thus, the second connecting rod 311 and the fourth connecting rod 312 can slide up and down to adapt to legs of different lengths. It should be noted that in other embodiments, similar slide grooves can also be provided in the thigh brace 210, and the third connecting rod 321 and the fifth connecting rod 322 can be provided in the corresponding slide grooves, so that the third connecting rod 321 and the fifth connecting rod 322 can also slide in the length direction of the thigh brace 210 to adjust the length of the extended thigh brace 210.

In another embodiment of the present application, the pivoting connection assembly 300 includes at least one hook and at least one hanging ring, and the at least one hook is arranged along the circumference of the calf brace 110, and the at least one hanging ring is arranged along the circumference of the thigh brace 210; the hook is fixedly connected to the thigh brace 210 through the hanging ring. For example, in one embodiment, a hook is arranged on the calf brace 110, and five hanging rings are arranged around the thigh brace 210, and the hook is located at the upper edge of the outer wall of the calf brace 110, and the five hanging rings are located at the lower edge of the outer wall of the thigh brace 210, and the five hanging rings are arranged and spaced apart around the thigh brace 210, so that when the calf brace 110 is put on, the hook can be hung on the corresponding hanging ring after the calf brace 110 is rotated inwardly or outwardly, and the hook and the hanging ring can also provide continuous traction to the calf brace 110. Different hanging rings for hanging represents different rotating angles. Users can select different hanging rings to hang the hooks on the calf brace 110 according to the degree of correction or rehabilitation required. Similarly, in another embodiment, two hooks can be set on the outer wall of the calf brace 110, and the two hooks are respectively located on the inner and outer sides of the calf. The connecting line of the two hooks passes through the axis of the calf brace 110. After the calf brace 110 rotates a certain angle, the inner and outer hooks are respectively hung on the corresponding hanging rings of the thigh brace 210. In addition, in other embodiments, the calf brace 110 can also be provided with more hooks, and the thigh brace 210 can also be provided with other numbers of hanging rings. Different hanging rings can be selected for hanging according to the required correction angle, which is convenient for users to use.

As shown in FIG. 1 and FIG. 4, in the embodiment of the present application, a first opening 130 is provided on the calf brace 110, and a second opening 230 is provided on the thigh brace 210. The first opening 130 runs through the entire side wall of the calf brace 110 along the length direction of the calf brace 110, and the second opening 230 runs through the entire side wall of the thigh brace 210 along the length direction of the thigh brace 210. The width of the first opening 130 and the second opening 230 can be set according to the user group. For example, the first opening 130 and the second opening 230 are respectively set to a quarter of the leg circumference, and the calf brace 110 and the thigh brace 210 are respectively three-quarters of the leg circumference of the calf and the thigh. On one hand, this setting is easier to wear, and on the other hand, straps 400 can be set on both sides of the opening. After the user puts on the calf brace 110 and the thigh brace 210, the volume of the calf brace 110 and the thigh brace 210 can be adjusted to a suitable size using the straps 400 according to the thickness of the user's calf and thigh.

It should be noted that the specific meaning of “generally” or similar words used in the embodiments of the present application is not ambiguous, but only means that the mechanical structure can be set according to the actual situation, and it does not need to be set completely according to a certain accuracy or standard. On the one hand, due to the complexity of the human body, some characteristics are not absolute. For example, the ankle joint mentioned above is not an absolute two-dimensional (planar) motion joint, but its range of motion as an omnidirectional motion joint is limited, so it can be regarded as a two-dimensional (planar) motion joint in the sense of the present application, so as to achieve the purpose of rotating the leg by rotating the direction of the foot. Similarly, since living people need to move and need comfort, it is not necessary to maintain 100% accuracy of a certain action during the correction and rehabilitation process, but only to maintain a certain action most of the time or in the sense of statistical average to achieve the purpose of rehabilitation or correction. Taking the ankle joint as an example, although the foot can be rotated to the left or right at a certain angle relative to the calf in the circumferential direction, normal people will comfortably place the foot in a position of 0 rotation, so the rotation angle of the first brace 100 in the embodiment of the present application can be based on this. In actual use, since the ankle joint still has a certain degree of flexibility, at a specific moment, the internal rotation or external rotation angle of the leg may deviate from the set value, but the statistical average is still within the set value. The same is true for the position of the plane formed by the left and right legs relative to the hip joint or the body.

The brace device in the embodiment of the present application is provided by setting the first brace 100, the second brace 200 and the pivotable connecting component 300, and the first brace 100 fixes the calf and the foot in the circumferential direction of the calf, the second brace 200 accommodates the thigh and is relatively fixed relative to the body, one end of the pivotable connecting component 300 is fixedly connected to the first brace 100, and the other end of the pivotable connecting component 300 is fixedly connected to the second brace 200. With the position of the pivot axis of the connecting component when toes are facing forward as a reference, the installation position of the connecting component 300 on the second brace 200 allows the pivot axis to rotate around the axis of the part of the second brace 200 that accommodates the thigh by an angle required for hip joint correction, so that the first brace 100 can force the calf to rotate the aforementioned angle relative to the toes facing forward, thereby driving the thigh and femoral head to rotate synchronously, and the second brace 200 is relatively fixed to the body. Therefore, the first brace 100 rotated relative to the second brace 200 can force the foot, calf, thigh and femoral head to rotate, that is, the femoral head can rotate the aforementioned angle in the hip joint socket, thereby playing a role in hip joint correction or rehabilitation.

The above description is only an optional embodiment of the present application, and does not limit the patent scope of the present application. All equivalent structural changes made by using the contents of the present application specification and drawings under the inventive concept of the present application, or directly/indirectly applied in other related technical fields are included in the patent protection scope of the present application.

What is claimed is:

1. A brace device for human hip joint correction, comprising:
a first brace for securing at least one side of a calf and a foot in a circumferential direction of the calf;
a second brace for accommodating at least one side of a thigh and securing the thigh relatively to a body; and
a pivotable connecting component, one end of which being fixedly connected to the first brace and the other end of which being fixedly connected to the second brace, and a pivot axis of the pivotable connecting component coinciding with a pivot axis of a knee joint when the brace device is put on;
wherein an installation position of the connecting component on the second brace is allowed to make the pivot axis rotate around an axis of the second brace and the thigh by an angle required for hip joint correction relative to a position of the connecting component when toes are facing straight ahead.

2. The brace device according to claim 1, wherein the first brace comprises a calf brace and a foot brace, the calf brace is used to be mounted outside the calf, the foot brace is used to be mounted outside the foot, and the calf brace is fixedly connected to the foot brace so that the calf brace is relatively fixed to the calf in the circumferential direction of the calf.

3. The brace device according to claim 2, wherein the second brace comprises a thigh brace and a fixing device, the thigh brace is used to be mounted outside the thigh, and the fixing device is used to fix the thigh brace relative to the body.

4. The brace device according to claim 3, wherein the fixing device comprises a hip brace and a connecting mechanism, the hip brace is used to be mounted on the human hip and fixed relative to the body; and
the connecting mechanism is used to connect the thigh brace to the hip brace.

5. The brace device according to claim 4, wherein the connecting mechanism comprises a first connecting rod and a movable joint, the movable joint is provided on the hip brace, one end of the first connecting rod is provided on the thigh brace, and the other end of the first connecting rod is provided on the movable joint; and
when the brace device is put on, the first connecting rod and the movable joint are located on left and right sides of the body, and are movably connected according to a preset degree of freedom, so that the thigh brace is allowed to move approximately in front and back directions relative to the hip brace.

6. The brace device according to claim 3, wherein the fixing device is a connecting member, the connecting member is used to connect the thigh braces of a left thigh and a right thigh on inner sides of legs, and the connecting member is rigidly connected to at least the thigh brace to be corrected.

7. The brace device according to claim 6, wherein the connecting member comprises a first connecting member and a second connecting member, one end of the first connecting member is connected to the thigh brace of the first side thigh, one end of the second connecting member is connected to the thigh brace of the second side thigh, and the other end of the first connecting member is connected to the other end of the second connecting member; and
an angle between the connecting member and the thigh brace determines a thigh abduction angle required for hip joint correction.

8. The brace device according to claim 7, wherein the first connecting member and the second connecting member are both provided with a plurality of threaded holes in a length direction, and the first connecting member and the second connecting member select the threaded holes required for a total length of the connecting member from the plurality of threaded holes for threaded connection.

9. The brace device according to claim 7, wherein a fixed connection position of one end of the first connecting member and the second connecting member connected to each other in the length direction is allowed to be adjusted steplessly.

10. The brace device according to claim 7, wherein the connecting member is connected to the thigh brace of the thigh on one side of the healthy hip joint through a universal joint.

11. The brace device according to claim 7, wherein a thigh abduction angle is allowed to be adjusted by adjusting an angle between the connecting member and the thigh brace.

12. The brace device according to claim 2, wherein the pivotable connecting assembly comprises a first assembly, the first assembly comprises a second connecting rod and a third connecting rod connected by a first pivot axis, the second connecting rod is fixedly connected to the calf brace, and the third connecting rod is fixedly connected to the thigh brace.

13. The brace device according to claim 12, wherein the pivotable connecting assembly further comprises a second assembly, the second assembly comprises a fourth connecting rod and a fifth connecting rod connected by a second pivot axis, the fourth connecting rod is fixedly connected to the calf brace, the fifth connecting rod is fixedly connected to the thigh brace, the first assembly and the second assembly are respectively located on inner and outer sides of a corresponding leg, and axes of the first pivot axis and the second pivot axis coincide.

14. The brace device according to claim 13, wherein positions of the third connecting rod and the fifth connecting rod in the circumferential direction of the thigh brace are adjustable.

15. The brace device according to claim 14, further comprising an arc-shaped slide rail coaxial with the thigh brace and arranged on an outer surface of the thigh brace, wherein ends of the third connecting rod and the fifth connecting rod connected to the thigh brace are respectively provided with a first slider, and the first slider is allowed to be fixedly connected to the arc-shaped slide rail at an adjustable position selected by an user.

16. The brace device according to claim 2, wherein the pivoting connection assembly comprises at least one hook and at least one hanging ring, the at least one hook is arranged along a circumference of the calf brace, and the at least one hanging ring is arranged along a circumference of the thigh brace; and
the hook is fixedly connected to the thigh brace through the at least one hanging ring.

17. The brace device according to claim 13, wherein an axis of the calf brace is located on a plane where the second connecting rod and the fourth connecting rod are located; and
an axis of the thigh brace is located on a plane where the third connecting rod and the fifth connecting rod are located.

18. The brace device according to claim 13, wherein the second connecting rod and the third connecting rod are telescopically connected to the calf brace in the length direction of the calf brace.

19. The brace device according to claim 18, wherein the calf brace is provided with two slide grooves in the length direction, and the second connecting rod and the fourth connecting rod are slidably arranged in the two slide grooves.

20. The brace device according to claim 19, wherein the second connecting rod and the fourth connecting rod are both provided with a plurality of through holes in the length direction, the calf brace is provided with a plurality of threaded holes corresponding to the plurality of through holes, and the second connecting rod and the fourth connecting rod are threadedly connected to the calf brace through the plurality of through holes and the plurality of threaded holes.
